(19) United States
(12) Patent Application Publication
(10) Pub. No.: US 2004/0176788 A1
(43) Pub. Date: Sep. 9, 2004

(54) Vacuum attachment system

(75) Inventor: Steven W. Opolski, Carlisle, MA (US)

Correspondence Address: TESTA, HURWITZ & THIBEAULT, LLP, HIGH STREET TOWER, 125 HIGH STREET, BOSTON, MA, 02110, US

(73) Assignee: NMT Medical, Inc., Boston, MA (US)

(21) Appl. No.: 10/384,042

(22) Filed: Mar. 7, 2003

(51) Int. Cl. A61B 17/32

(52) U.S. Cl. 606/167

(57) ABSTRACT

A medical device comprising a flexible, elongated member sized and shaped to fit within a vessel and defining a first lumen. A distal end of the flexible, elongated member including a port for connection to a vacuum source. A wire is slideably positionable within the first lumen and is movable between a first position and a second position. A cutting instrument is attached to a distal end of the wire and is retracted within the distal end of the lumen when the wire is at the first position and extending outside the distal end of the lumen when the wire is at the second position.

### FIELD OF THE INVENTION

This invention relates generally to a method and apparatus for repairing cardiac defects and more specifically to a method and apparatus for temporality immobilizing a local region of heart tissue thereby permitting an interventional procedure to be performed.

### DESCRIPTION OF THE RELATED ART

Numerous systems for permitting percutaneous procedures have been devised over the years in order for physicians to perform procedures such as the delivery and positioning of implants within the human body in a minimally invasive manner. An example of one such percutaneous procedure involves the placement of a device within the cardiac septum to correct a defect in the septum. To understand the difficulties associated with this procedure it is first necessary to discuss the anatomy of the human heart.

The human heart is divided into four compartments or chambers. The left and right atria are located in the upper portion of the heart and the left and right ventricles are located in the lower portion of the heart. The left and right atria are separated from each other by a muscular wall, the intraatrial septum, while the ventricles are separated by the intraventricular septum. Either congenitally or by acquisition, abnormal openings, holes or shunts can occur between the chambers of the heart or the great vessels, causing shunting of blood through the opening. These holes or shunts may develop between the left and right atria along the muscular wall which separates the two: the intraatrial septum.

Such deformities are usually congenital and result from the incomplete formation of the septum, or wall, between chambers during fetal life when the heart forms from a folded tube into a four chambered, two unit system. These deformities can cause significant problems which ultimately, if uncorrected, cause added strain on the heart which in turn may result in heart failure. One such defect, a patent foramen ovale (PFO), is a persistent, one-way, usually flap-like opening in the wall between the right atrium and left atrium of the heart. The PFO is the most common abnormality of fetal origin among the normal adult population. The opening between the right atrium and left atrium is formed because the embryonic left-sided septum primum is thinner than the embryonic septum secundum and overlaps the septum secundum. Since left atrial (LA) pressure is normally higher than right atrial (RA) pressure, the flap typically stays closed. Under certain conditions, however, RA pressure can exceed LA pressure creating the possibility for right to left shunting that can allow blood clots to enter the systemic circulation. This is of particular importance with patients who are prone to forming venous thrombus such as those with deep vein thrombosis or clotting abnormalities.

Transcatheter (percutaneous) closure of PFOs has become possible using a variety of mechanical closure devices, allowing patients to avoid the potential side effects often associated with standard anticoagulation therapies. These devices consist of a metallic structural framework combined with a synthetic material. The function of the material is to encourage ingrowth and encapsulation of the implant by a fibrous tissue capsule covered by a neointima. Other intracardiac defects also currently treated with such devices include atrial septal defects (ASDs), ventricular septal defects (VSDs), and left atrial appendages (LAAs).

The conventional technique for closure of a PFO using the transcatheter method involves the placement of the device across the oblique-angled tunnel-like pathway of the PFO through the septum. Unfortunately, the tortuosity of the opening may result in the deformity of the closure device when introduced through the PFO tunnel. The physician may therefore decide to position the device over the PFO by producing a new less tortuous opening in the septum. This procedure is defined as a septal puncture.

Such an interventional procedure requires that a physician establish and maintain a position of the delivery device at a particular location in the body, such as at the wall of the septum, substantially during the entire procedure. This is particularly problematic when the target location for the interventional procedure is in motion due to muscular contractions, such as due to the beating of the heart. The tissue is also prone to deflecting (or tenting) due to a localized force (such as by the tip of a lance) applied to the tissue surface. The tissue motion and/or tendency of the tissue to deflect after the puncture is made may result in damage to adjacent or surrounding tissue near the puncture site by the device used to create the puncture.

The present invention provides a method and apparatus for locating and maintaining the position of a device and preventing the tenting of the tissue at a given position in the body.

### SUMMARY OF THE INVENTION

The invention relates to a system that combines a flexible elongate member and a suction source so that the flexible elongate member, such as a catheter, removably attaches to a target tissue location. The invention, in one exemplary embodiment, is useable when a physician needs to puncture the atrial septum using a lance or needle.

In one embodiment, the system includes a device which includes a flexible elongate member with a first lumen extending therethrough. The flexible elongate member and the first lumen include a proximal end and a distal end. The flexible elongate member is sized and shaped to fit within a blood vessel of the body. The proximal end of the flexible elongate member includes a port for connection to a vacuum source (e.g., pump, syringe, aspirator or bulb). A wire having a proximal end and a distal end is positioned within the first lumen. The wire is slideably movable between a first position and a second position.

The device further includes a lance attached to the distal end of the wire. The lance is retracted within the distal end of the lumen when the wire is at the first position, and the lance is extended outside the distal end of the lumen when the wire is at the second position. In one embodiment, the lance is sized and shaped to puncture the septum of the heart thereby allowing insertion of the occluder.

In one embodiment, the system includes a vacuum source in communication with the port of the elongate member for establishing at least a partial vacuum in the first lumen at the distal end of the elongate member. In other embodiments, the device includes a second lumen having a proximal end and a distal end and a vacuum source in communication with the port of the elongate member for establishing at least a partial vacuum in the second lumen at the distal end of the elongate member.

In one embodiment, the device also includes a handle attached to the proximal end of the elongate member and an actuator at least partially disposed in the handle for moving the wire between the first position and the second position.

In another aspect, the invention relates to a method for repairing a cardiac defect. The method includes the step of providing a medical device having a flexible elongate member with a first lumen extending therethrough. The flexible elongate member and the first lumen have a proximal end and a distal end. The flexible elongate member is sized and shaped to fit within a vessel, and the proximal end of the flexible elongate member has a port for connection to a vacuum source. A wire slideably movable within the first lumen and the wire has a proximal end and a distal end. The wire is movable between a first position and a second position. A lance is attached to the distal end of said wire. The lance being retracted within the distal end of the lumen when the wire is at said first position and the lance is extended outside the distal end of the lumen when the wire is at the second position. The lance is sized and shaped to puncture the septum of the heart to allow insertion of an occluder. The method further includes the steps of inserting the distal end of the flexible elongate member into the body of the patient; activating the vacuum source to reversibly adhere the distal end of the lumen to the target tissue; advancing the lance into the target tissue thereby creating a puncture or opening; inserting a guide member through the puncture; inserting an implant into the puncture; and removing the distal end of the lumen from the body of the patient.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic side-view of one embodiment of the vacuum attachment system according to the invention;

FIG. 2A is a detailed sectional view of the distal end of the system of FIG. 1 depicting the lance in a retracted position;

FIG. 2B is a detailed sectional view of the distal end of the system of FIG. 1 depicting the lance in an extended position;

FIGS. 3A-3C are schematic views of a clinical application of the vacuum attachment system embodiment depicted in FIG. 1; and

FIGS. 4A and 4B are detailed perspective views of the distal end of another embodiment of the system of FIG. 1.

### DESCRIPTION OF THE PREFERRED EMBODIMENT

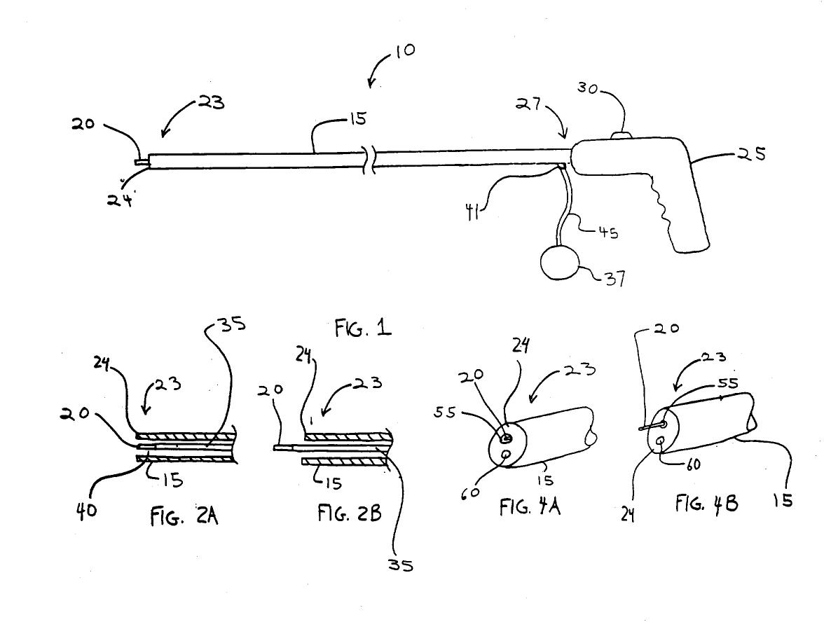

Referring to FIG. 1 and FIG. 2A, one embodiment of the medical device 10 is shown according to the invention. The device 10 includes a flexible elongate tubular member 15, such as a catheter, which is sized and shaped to fit within the blood vessel of a body. The flexible elongate tubular member 15 defines a lumen 40 within which is located a wire 35 which is longitudinally movable within the lumen 40. A lance 20 or other cutting device located at a distal end 23 of the wire 35. The proximal end of the wire 35 extends through the proximal end 27 of the flexible elongate tubular member 15. A handle assembly 25 is located at a proximal end 27 of the flexible elongate tubular member 15 and includes an actuator 30. The actuator 30 is disposed within the handle 25 and is in physical communication with the wire 35. Use of the actuator 30 imparts relative movement between the wire 35 and the flexible elongate tubular member 15 as known to one skilled in the art.

Referring to FIGS. 2A and 2B, in one embodiment, when the actuator 30 is not engaged, the wire 35 and lance 20 are retracted within the lumen 40 of the flexible elongate tubular member 15. When the actuator 30 is engaged, the wire 35 and lance 20 extend from the lumen 40 of the flexible elongate tubular member 15. Thus if the distal end or rim 24 of the flexible elongate tubular member 15 is placed against tissue when the actuator 30 is engaged, the lance 20 will penetrate the tissue.

In one embodiment, a vacuum source 37 is connected to the proximal end 27 of the flexible elongate tubular member 15 at a suction port 41. The suction port 41 is adapted for receiving one end of a vacuum tube 45. The opposite end of the vacuum tube 45 is attached to the vacuum source 37. Operation of the vacuum source 37 establishes at least a partial vacuum within the lumen 40 of the flexible elongate tubular member 15.

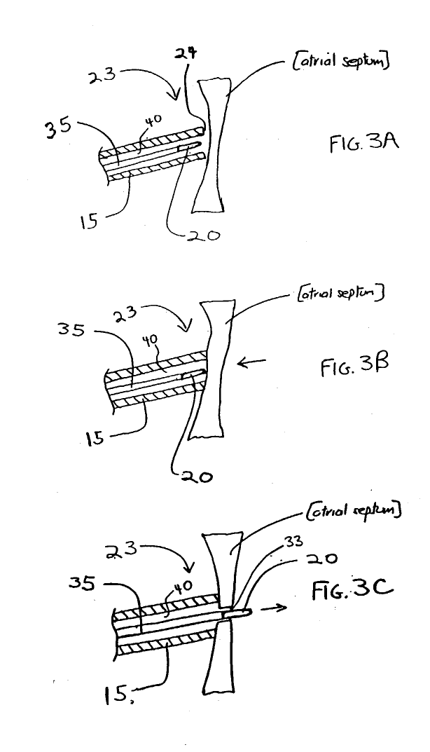

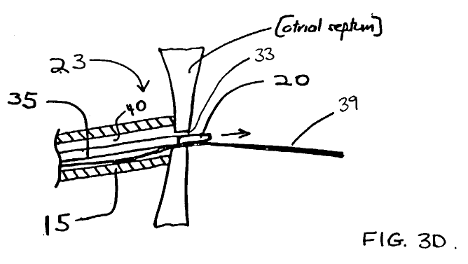

Referring also to FIGS. 3A-3C, during use the clinician positions the distal end 23 of the flexible elongate tubular member 15 into a vessel of the patient and moves the end of the flexible elongate tubular member 15 to the location of the procedure. In the example shown, the location is the atrial septum, but any location could be used. When the distal end 23 or rim 24 of the flexible elongate tubular member 15 is placed against the atrial septum, as shown in FIG. 3B, the vacuum source 37 is activated to establish at least a partial vacuum within the lumen 40. The partial vacuum releasably secures the treatment site of the atrial septum proximal to the rim 24 of the flexible elongate tubular member 15. Referring to FIG. 3C, at this time the actuator 30 is engaged and the wire 35 moves within the lumen 40 so as to cause the lance 20 to extend from the lumen 40 and create a puncture 33 in the septum while the rim 24 is held fast against the septum by the vacuum created by the vacuum source 37. The action of the vacuum holding fast the septum during creation of the puncture may aid with minimizing deflection (or tenting) of the tissue in proximity to the puncture. Referring now to FIG. 3D, a guide member 39 is then extended through the puncture 33 in the septum to, for example, maintain the position of the distal end 23 of the flexible elongate member 15 with respect to the puncture 33. The flexible elongate member 15 may, for example, then be retracted from the body of the patient and a septal occluder may be inserted into the puncture in the septum primum of the heart. By way of example, the septal occluder may be a CardioSEAL® medical device manufactured by NMT Medical, Inc. (Boston, Mass.).

The guide member 39 might be, for example, a guidewire, catheter or tubular member that is suitable for introduction through the lumen 40 of the flexible elongate tubular member 15 and through an opening or puncture created by the lance 20.

The lumen 40 may, alternatively, be employed to measure pressure in the body by means of a pressure transducer connected to the lumen 40 external to the body of the patient.

FIGS. 4A and 4B depict an alternative embodiment in which the tubular member 15 includes two lumen, a first lumen 55 and a second lumen 60. The first lumen 55, carries the wire 35 and lance 20 while the second lumen 60 delivers the vacuum application to the distal end 23 of the flexible elongate tubular member 15.

One skilled in the art will realize that variations are possible that would not deviate from the spirit of the invention. For example although the actuator 30 is shown as a button on the handle 25, the actuator 30 could alternatively in the form of a trigger or some other configuration. Similarly, although a lance 20 is depicted a cutting, puncturing or biopsy device could be used in the device. Alternative numbers (e.g., one, two, three, etc.) and configurations (e.g., distributed in a radial pattern or in a 2×2 array relative to the face of the rim 24 of the flexible elongate tubular member 15) of lumens are contemplated that are capable of fixing the position of the distal tip 23 of the flexible elongate tubular member 15 relative to the tissue 50 before, during, and after a puncture is created in the tissue 50. It is also contemplated that a plurality of lumens may be capable of creating a vacuum relative to the surface of the tissue.

While the present invention has been described in terms of certain exemplary preferred embodiments, it will be readily understood and appreciated by one of ordinary skill in the art that it is not so limited and that many additions, deletions and modifications to the preferred embodiments may be made within the scope of the invention as hereinafter claimed. Accordingly, the scope of the invention is limited only by the scope of the appended claims.

What is claimed is:

1. A medical device for removably holding a tissue while a procedure is performed, said medical device comprising:
a flexible elongate member defining a first lumen extending therethrough, said flexible elongate member and said first lumen having a proximal end and a distal end, said flexible elongate member being sized and shaped to fit within a vessel, and the lumen of said elongate member in communication with a port for connection to a vacuum source;
a wire slideably positionable within said first lumen, said wire having a proximal end and a distal end, and said wire being movable between a first position and a second position; and
a cutting device attached to the distal end of said wire, said cutting device being retracted within the distal end of the lumen when the wire is at said first position, said cutting device being extended outside the distal end of said lumen when the wire is at said second position.

2. The medical device of claim 1 wherein said cutting device is a lance being sized and shaped to puncture a septum of the heart to allow insertion of the occluder.

3. The medical device of claim 1 further comprising a source in communication with said port of said elongate member, said source establishing at least a partial vacuum in said first lumen at the distal end of said elongate member.

4. The medical device of claim 1 further comprising:
a second lumen having a proximal end and a distal end; and
a port in communication with said second lumen,
wherein said source is in communication with said port of said second lumen, and
wherein said source establishing at least a partial vacuum in said second lumen.

5. The medical device of claim 1 further comprising a handle attached to the proximal end of said elongate member.

6. The medical device of claim 5 further comprising an actuator for moving said wire between said first position and said second position.

7. The medical device of claim 6 further comprising an actuator at least partially disposed in said handle for moving said wire between said first position and said second position.

8. A method for performing a procedure on a tissue, said method comprising the steps of:
a. providing a medical device comprising:
(i) a flexible elongate member with a first lumen extending therethrough, said elongate member and said first lumen having a proximal end and a distal end, said elongate member being sized and shaped to fit within a vessel, and the lumen of said elongate member in communication with a port for connection to a source,
(ii) a wire slideably positionable within said first lumen, said wire having a proximal end and a distal end, and said wire being movable between a first position and a second position,
(iii) a cutting device attached to the distal end of said wire, said cutting device being retracted within the distal end of the lumen when the wire is at said first position and being extended outside the distal end of said lumen when the wire is at said second position
b. inserting the distal end of the flexible elongate member into the body of the patient and moving it to the tissue interest;
c. activating said source to reversibly adhere the distal end of said lumen to the tissue of interest;
d. advancing said cutting device into the target tissue creating an opening in said tissue;
e. advancing a guide member through the opening in said tissue; and
f. removing the distal end of the flexible elongate member from the body of the patient.
